(19) United States
(12) Patent Application Publication
(10) Pub. No.: US 2008/0280902 A1
(43) Pub. Date: Nov. 13, 2008

(54) Inhibitors of Checkpoint Kinases

(76) Inventors: Mark E. Fraley, North Wales, PA (US); Robert M. Garbaccio, Lansdale, PA (US); Shaei Y. Huang, Lansdale, PA (US); Justin T. Steen, Harleysville, PA (US)

Correspondence Address: MERCK AND CO., INC, P O BOX 2000, RAHWAY, NJ, 07065-0907, US

(21) Appl. No.: 11/795,005

(22) PCT Filed: Jan. 24, 2006
(86) PCT No.: PCT/US06/02396

§ 371 Date: Jul. 10, 2007

Related U.S. Application Data
(60) Provisional application No. 60/647,997, filed on Jan. 28, 2005.

(51) Int. Cl. A61K 31/5377; C07D 401/04; A61K 31/497; C07D 401/14; A61P 35/00; C07D 413/14; A61K 31/4709

(52) U.S. Cl. 514/235.5; 546/156; 514/312; 514/253.08; 544/128; 544/363

(57) ABSTRACT

The instant invention provides for compounds which comprise substituted quinolinones that inhibit CHK1 activity. The invention also provides for compositions comprising such inhibitory compounds and methods of inhibiting CHK1 activity by administering the compound to a patient in need of treatment of cancer.

### BACKGROUND

1. Technical Field

The present invention relates to the field of telecommunications and digital computing. More specifically, the invention relates to the field of digital computing in both an online and offline states of connectivity.

2. Discussion of Related Technology

The Internet has achieved such widespread use among ordinary people that many wonder how they ever lived without the ease and convenience of having so much information so readily available. Today, anyone with an Internet connection can easily scour endless sources of information on everything from the latest breaking news to the most outdated and obscure facts ever committed to writing. New sites and sources of information are being created almost more quickly than they can be counted.

The simplicity with which information is made available over the Internet has even spilled over into other areas. For instance, today many employers make employment information, such as human resources data, available over their corporate networks. Many schools are beginning to provide online access to the schools' libraries over the schools' private network. Other examples are numerous.

This overwhelming availability of knowledge has naturally generated avid followers, addicts almost. The simplicity with which users can access scores of information has, also naturally, led to its commercialization. People that avail themselves of a Web site that offers the latest news update may are be subjecting themselves to electronic advertising. In other words, information, such as news articles and the like, is often presented to a user along side an image or other form of electronic advertisement. The information providers are being paid for displaying or presenting the electronic advertisement, which enables the provider to make the information itself available for free or nearly free. In this way, the online frontier is mimicking the business models of network television, commercial radio, and most magazines.

However, there are new problems, challenges, and even advantages that face information providers and advertisers in the online world. For instance, how are users targeted or advertised to using this technology when not online? The nature of this technology is often such that the information can be presented in many different formats without affecting the information content itself. In other words, a news article is generally no more or less informative in two columns rather than one. This reality has opened a world of options to the providers of information when choosing how best to advertise to the consumer. Similarly, the ability of information providers to track or monitor patterns with which information is consumed allows targeted advertising in a way never before possible.

A system for electronic advertising that is worthy of today's technology has eluded those skilled in the art, until now.

### SUMMARY

This summary is provided to introduce a selection of concepts in a simplified form that are further described below in the Detailed Description section. This summary is not intended to identify key features or essential features of the claimed subject matter, nor is it intended to be used to limit the scope of the claimed subject matter.

According to aspects of various described embodiments, implementations are provided for enabling offline electronic advertisement. Briefly stated, a client computer is enabled to view content offline, and electronic advertisements may be presented in conjunction with the content. Usage data is tracked for the advertisements and reported to an ad server upon synchronization so that appropriate steps can be taken (i.e., billing a customer) based on the usage of the advertisements. The advertisements are selected and presented in conjunction with content offline using a dynamic layout system in which the content and the advertisements are presented in a visually appealing manner.

### BRIEF DESCRIPTION OF THE DRAWINGS

Many of the attendant advantages of this invention will become more readily appreciated as the same becomes better understood by reference to the following detailed description, when taken in conjunction with the accompanying drawings, briefly described here.

FIG. 1 is a functional block diagram of a network environment in which implementations of the invention are particularly applicable.

FIG. 2 is a functional block diagram generally illustrating an ad server as introduced above in conjunction with FIG. 1.

FIG. 3 is a functional block diagram generally illustrating components of the client computer introduced above in conjunction with FIG. 1.

FIG. 4 is a functional block diagram generally illustrating the client computer with greater detail on the presentation component introduced above in conjunction with FIG. 2.

FIG. 5 is a functional block diagram of an exemplary computing device that may be used to implement one or more parts of the system described above.

FIG. 6 is an operational flow diagram generally illustrating a process for presenting an electronic advertisement on a client computer.

FIG. 7 is an operational flow diagram generally illustrating a process for presenting an advertisement on a client computer.

The invention will now be described in detail with reference to these Figures wherein like numerals refer to like elements throughout.

### DETAILED DESCRIPTION

Various embodiments are described more fully below with reference to the accompanying drawings, which form a part hereof, and which show specific exemplary implementations for practicing various embodiments. However, other embodiments may be implemented in many different forms and should not be construed as limited to the embodiments set forth herein; rather, these embodiments are provided so that this disclosure will be thorough and complete. Embodiments may be practiced as methods, systems or devices. Accordingly, embodiments may take the form of a hardware implementation, an entirely software implementation, or an implementation combining software and hardware aspects. The following detailed description is, therefore, not to be taken in a limiting sense.

The logical operations of the various embodiments are implemented (1) as a sequence of computer implemented steps running on a computing system and/or (2) as interconnected machine modules within the computing system. The implementation is a matter of choice dependent on the performance requirements of the computing system implementing the embodiment. Accordingly, the logical operations making up the embodiments described herein are referred to alternatively as operations, steps or modules.

Briefly stated, a client computer is enabled to view content offline, and electronic advertisements may be presented in conjunction with the content. Usage data is tracked for the advertisements and reported to an ad server upon synchronization so that appropriate steps can be taken (i.e., billing a customer) based on the usage of the advertisements.

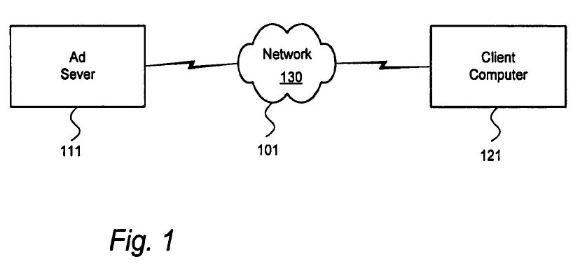

FIG. 1 is a functional block diagram of a network 101 environment in which implementations of the invention are particularly applicable. As illustrated, a remote advertisement server (ad server 111) and a client computer 121 are coupled to a wide area network 101, such as the Internet. The ad server 111 is a computing system that is used to provide or make available advertisements used in electronic advertising, such as in connection with Web pages that are viewed while browsing the Web. Electronic advertising is familiar to most users of the Internet. The ad server 111 may additionally include content that can be viewed by other computers coupled to the network 101.

Generally stated, the client computer 121 includes components that enable users to browse information or content provided over the network 101. Examples of the type of content that can be viewed while online are endless, and include news reports, stock quotes, streaming media (e.g., music and video), and the like. Generally, the client computer 121 retrieves content from various sources, such as the ad server 111, over the network 101. In this particular implementation, the client computer 121 is also configured to enable content to be viewed offline, such as by downloading and caching content to the client computer 121. Moreover, electronic advertisements that may be presented in conjunction with the cached content are also downloaded and cached at the client computer 121.

As will be described in greater detail below, a user may take the client computer 121 offline, yet still view the cached content and the cached advertisements. In addition, the client computer 121 tracks which advertisements are presented to the user, and reports that information to the ad server 111. In this way, the ad server 111 can make electronic advertisements available even while the client computer 121 is offline, yet the ad server 111 can continue to track presentation data of the advertisements, such as for the purpose of billing the advertisers.

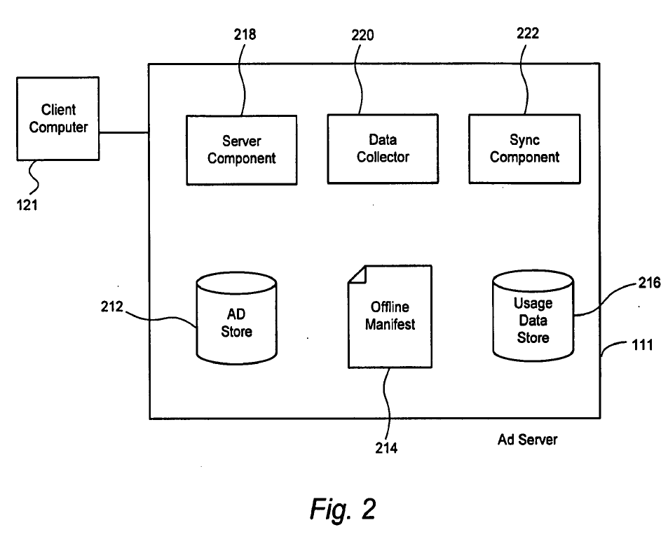

FIG. 2 is a functional block diagram generally illustrating an ad server 111 as introduced above in conjunction with FIG. 1. In this particular implementation, the ad server 111 includes several components that support electronic, online advertising. The ad server 111 may include many other components in addition to those described here, but which are not relevant to the present discussion.

The ad server 111 includes an ad store 212, which is a storage component in which resides electronic advertisements, such as images, multimedia files, scripts, and the like. Each advertisement may be associated with a customer, which is any entity that may provide advertisements to the ad server 111 for presentation, or may request the presentation of an advertisement on its behalf. Commonly, the customer agrees to provide compensation to the ad server 111 operator based on a number of times its associated advertisements are presented.

The ad server 111 also includes a server component 218, which is an executable component that is configured to respond to requests for an electronic advertisement by retrieving the requested advertisement from the ad store 212 and transmitting it to the requesting entity. The request may take the form of an HTTP request, or the like, issued in connection with viewing a Web page or other markup language content.

A data collector 220 is an executable component of the ad server 111 that is configured to monitor the usage of or requests for an advertisement. In this particular implementation, the data collector 220 keeps track of each time an advertisement is accessed or requested. More specifically, the data collector 220 may track each request to retrieve an advertisement from the ad server 111, and any ‘click-through’ information for each advertisement. In other words, while an advertisement is being presented or displayed, a user may select or click on the advertisement to investigate the subject matter of the advertisement. Because there may be more value to a customer when users click on an advertisement than simply view it, the data collector 220 tracks that information, such as for billing purposes.

A usage data store 216 on the ad server 111 is a data storage component in which is stored the usage data collected by the data collector 220. Examples of the types of data that may be stored include the number of times each advertisement has been presented or retrieved, the number of times a user followed a link associated with advertisement, other empirical data for each advertisement, and the like.

An offline manifest 214 resides on the ad server 111. The offline manifest 214 is a data file or data stream that identifies a set of advertisements that should be available at a client computer 121 when the client computer 121 is offline. In one implementation, the offline manifest 214 is an eXtensible Markup Language (XML) that includes identifying information for each advertisement that is to be available offline, and may include the advertisement itself. Examples of the identifying information include a filename (or the like) for the advertisement, physical dimensions of the advertisement (e.g., height and width), a size of the advertisement (e.g., in bytes), a location (e.g., a URL or URI), and the like.

A sync component 222 of the ad server 111 is an executable component configured to facilitate offline ad serving on a client computer 121. The sync component 222 receives an inbound communication indicating that a client computer 121 desires to initiate or prepare for offline usage. In response, the sync component 222 performs functions to support the offline usage, including providing the offline manifest 214 to the client computer 121 and perhaps receiving any offline usage data that the client computer 121 may provide. However, as described in greater detail below, this particular implementation of the invention mitigates the need for modifications to the ad server 111 to support offline usage by imitating, during synchronization, the usage that would have been experienced at the ad server 111 if the client computer 121 had been online.

The components described here are merely illustrative of the type and nature of components that may be used on the ad server 111 to implement the techniques and functionality just described. In alternative embodiments, other components may be used, certain functions may be combined into fewer components, certain functions may be divided among different components, additional functions may be incorporated into these or other components, and the like.

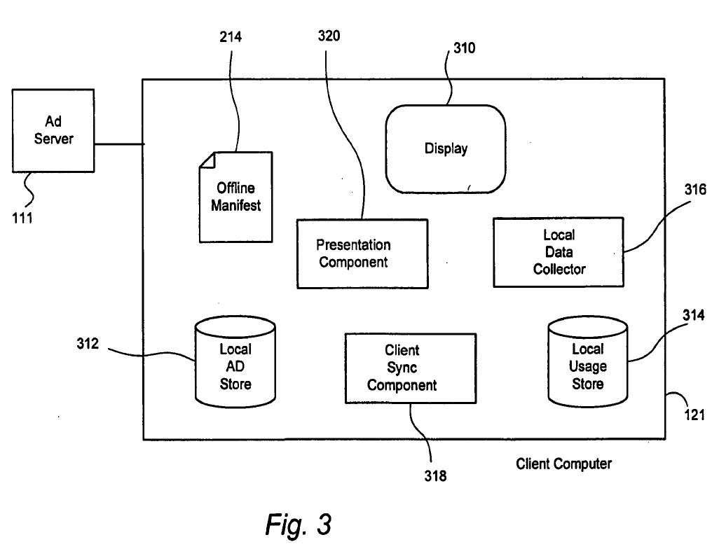

FIG. 3 is a functional block diagram generally illustrating components of the client computer 121 introduced above in conjunction with FIG. 1. In this particular implementation, the client computer 121 includes several components that enable offline content viewing and electronic advertising. In accordance with this implementation, the components enable offline advertising without compromising the economic advantages of online advertising.

The client computer 121 includes a copy of the offline manifest 214 retrieved from the ad server 111. As will be discussed, the offline manifest 214 may be delivered to the client computer 121 at most any time, such as during a synchronization when the client computer 121 goes online, or during an initialization procedure where the client computer 121 is being prepared for offline use. It should be appreciated that the offline manifest 214 need not be delivered during an online session, but instead could be delivered in some offline manner, such as on an installation medium, or the like.

A local ad store 312 is also included on the client computer 121. The local ad store 312 is a data storage component in which reside local copies of advertisements for presentation on the client computer 121. For example, the local ad store 312 may include copies of each of the advertisements identified in the offline manifest 214.

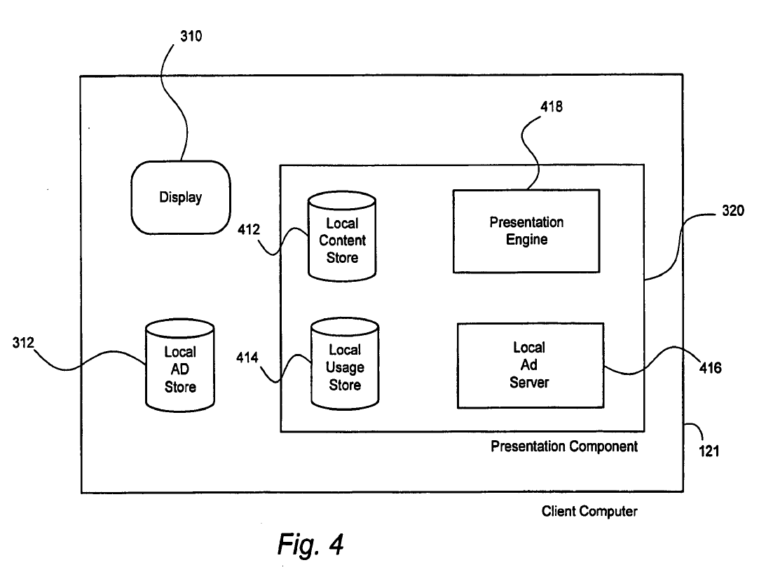

A presentation component 320 on the client computer 121 is configured to generate and display content on a display 310. The presentation component 320 is described in greater detail below in conjunction with FIG. 4. Briefly stated, the presentation component 320 is configured to retrieve content, such as markup language pages and media content, and to present the content on a local display 310 using a content-appropriate layout. In accordance with this implementation, the presentation component 320 is configured to include advertisements in conjunction with other content being displayed. For example, the presentation component 320 may be configured to present to a user certain articles pertaining to a topic.

A local data collector 316 on the client computer 121 is a component that performs substantially the same operations as the data collector 220 resident on the ad server 111 (FIG. 2) except on the client computer 121 while it is offline. In other words, the local data collector 316 interacts with the presentation component 320 to determine usage data about each of the advertisements that are presented on the mobile device while the client computer 121 is offline. The usage data collected may include the same information as that collected by the ad server data collector 220, or it may contain less or more information. The usage data collected by the local data collector 316 is stored in a local usage store 314, which may be any storage component for persistently storing data between online sessions.

In this implementation, the client computer 121 includes a client sync component 318 that is responsible for enabling proper offline support of electronic advertising. More specifically, the client sync component 318 is responsible for and configured to retrieve the offline manifest 214 from the ad server 111 during a synchronization event. In addition, the client sync component 318 may be configured to retrieve the offline manifest 214 from a location other than the ad server 111. In conjunction with retrieving the offline manifest 214, the client sync component 318 may additionally retrieve a copy of each advertisement referred to in the offline manifest 214 and save those copies in the local ad store 312.

The client sync component 318 is further configured to provide the local usage data to the ad server 111 during a synchronization event. Although there are many different possible techniques for providing the local usage data, this implementation of the invention seeks to mitigate any modifications to the remote ad server 111. Accordingly, the client sync component 318 essentially recreates all (or substantially all) of the operations, such as advertisement retrievals and click throughs, that were performed while the client computer 121 was offline. In other words, during a subsequent synchronization, the client sync component 318 refers to the local usage data and, for example, issues a request to retrieve each advertisement from the ad server 111 an equal number of times the advertisement was retrieved from the local ad store 312 212 while the client computer 121 was offline. The client sync component 318 performs these operations to mimic to the ad server 111 the usage that has occurred on the client computer 121 while offline. Alternatively, the local usage data could be delivered to the ad server 111 in some other manner, perhaps as a data file that includes itemized usage information for each local advertisements. In such an alternative, the ad server 111 would require additional components to parse and consume such a data file in a meaningful way.

FIG. 4 is a functional block diagram generally illustrating the client computer 121 with greater detail on the presentation component 320 introduced above in conjunction with FIG. 2. As illustrated in FIG. 4, the client computer 121 includes several components to facilitate the presentation of content in conjunction with advertisements while the client computer 121 is offline. These components are selected and configured to dynamically layout the content and any advertisements in such a way that the end result is spatially appealing. The desired result is an appearance that the content and advertisement were organized in advance and simply rendered in a previously-optimized layout.

The components that are provided to pursue this goal include the local ad store 312 as described above in conjunction with FIG. 3, and a display 310 on which data may be rendered, displayed, presented, or the like. In one example, the display 310 may be a window associated with browsing software components or the like. The display 310 has display characteristics, such as width, height, color depth, and the like. These display characteristics may also change periodically, such as if a user resizes the window.

Another component of the client computer 121 is the presentation component 320, introduced above. In this particular implementation, the presentation component 320 includes a local content store 412 in which resides content that is to be rendered, presented, or displayed on the client computer 121. Examples of the content are limitless, and generally include any form of data that can be displayed to a user of the client computer 121. For instance, the content may include articles or text, such as the news, multimedia content, any form of markup based information, weather and/or stock reports, financial or business updates, images, and the like. One common characteristic of the content is that the content is amenable to presentation in a dynamic layout environment.

The presentation component 320 further includes a local template store 414 in which reside a number of layout templates. In this particular implementation, the content is amenable to presentation in different visual formats. For instance, the content is a textual news article, the content may be presented in single column or multiple column format. The content may be a news article that includes an image, in which case the content may benefit from a two column layout or a layout that includes an image area. The templates are pre-defined layout descriptions that define the characteristics that will be used to present content. For example, one template may define a single column textual layout to display text in one column. Another template may define a dual column textual layout to display text in two columns. The templates may also define image areas within the layout in which either an image or other multimedia content may be presented. Moreover, the templates may include regions, both textual and non-textual, that are reserved for or in which advertisements are preferred.

A presentation engine 418 is also provided in the presentation component 320. The presentation engine 418 is configured to interact with the other components to generate a complete layout for presentation on the display 310. In this implementation, the presentation engine 418 determines, based on certain criteria, which of the layout templates is most appropriate for presenting the content currently being presented on the display 310. The selection criteria that the presentation engine 418 may use includes the current display characteristics of the display 310, the nature and characteristics of the content being presented, and perhaps whether an advertisement should be or needs to be presented. Other selection criteria may also be used.

Once an appropriate template is selected, the presentation engine 418 lays out the current content using the selected template. In many cases, “white space” may remain where the content does not perfectly or completely fill out the selected template. In certain cases, the template may have been selected to leave a certain amount of white space, perhaps at a particular location in the layout. In either event, the presentation engine 418 may request an advertisement to present in that white space. As will be discussed next, the presentation engine 418 may request an advertisement from the local ad server 416. The content and any advertisements are then presented on the display 310.

A local ad server 416 is a component that is responsible for and configured to serve advertisements from the local ad store 312 to the presentation engine 418. In this implementation, because of the dynamic nature of content layout, the local ad server 416 may include logic and functionality to select from various advertisements based on various selection criteria. For example, the presentation engine 418 may request an advertisement from the local ad server 416 that meets certain dimensions, such as size and aspect ratio. The local ad server 416 may examine each of the locally stored advertisements to select one that satisfies the selection criteria. It should be noted that the advertisements may be specified with ranges for certain criteria, and the local ad server 416 may compare the selection criteria to the entire ranges associated with each advertisement. For instance, a particular advertisement may specify that it can properly be displayed at a size between a minimum and a maximum width and/or height. In addition, the advertisement may specify that its aspect ratio can be altered within some range. These and other criteria may be used by the local ad server 416 to select an advertisement.

It should be appreciated that the local ad server 416 may additionally be configured to force the use of one or more advertisements. In other words, the local ad server 416 may be configured with instructions to force the presentation of one or more advertisements. For instance, a certain advertiser may pay a premium to ensure that its advertisements are presented a minimum number of times, or the like. In such a case, the local ad server 416 may be configured to promote one or more advertisements over the others. These and other alternatives will become apparent to those skilled in the art.

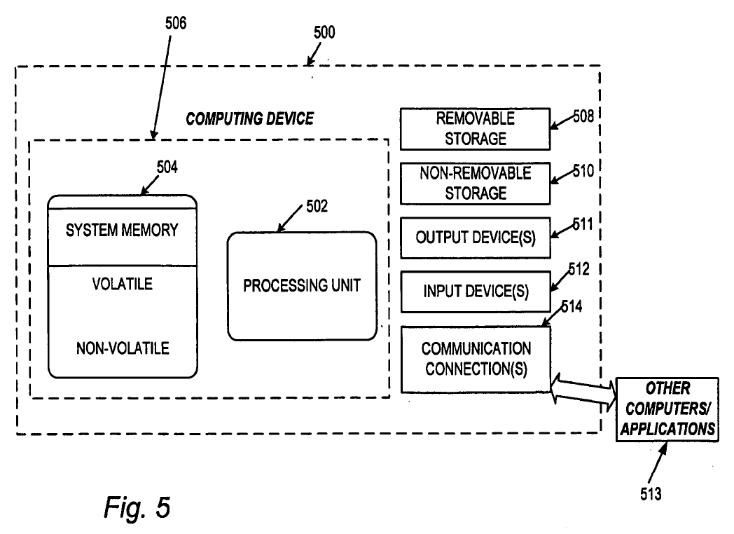

FIG. 5 is a functional block diagram of an exemplary computing device 500 that may be used to implement one or more parts of the system described above. The computing device 500, in one basic configuration, includes at least a processing unit 502 and memory 504. Depending on the exact configuration and type of computing device, memory 504 may be volatile (such as RAM), non-volatile (such as ROM, flash memory, etc.) or some combination of the two. This most basic configuration is illustrated in FIG. 5 by dashed line 506.

Additionally, device 500 may also have additional features/functionality. For example, device 500 may also include additional storage (removable and/or non-removable) including, but not limited to, magnetic or optical disks or tape. Such additional storage is illustrated in FIG. 5 by removable storage 508 and non-removable storage 510. Computer storage media includes volatile and nonvolatile, removable and non-removable media implemented in any method or technology for storage of information such as computer readable instructions, data structures, program modules or other data. Memory 504, removable storage 508 and non-removable storage 510 are all examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can accessed by device 500. Any such computer storage media may be part of device 500.

Computing device 500 includes one or more communication connections 514 that allow computing device 500 to communicate with one or more computers and/or applications 513. Computing device 500 may also have input device(s) 512 such as keyboard, mouse, pen, voice input device, touch input device, etc. Output device(s) 511 such as a monitor, speakers, printer, PDA, mobile phone, and other types of digital display 310 devices may also be included. These devices are well known in the art and need not be discussed at length here.

What follows are descriptions of generalized processes that implement various, non-exclusive embodiments of the invention. These processes may be implemented using computer-executable instructions in software or firmware, but may also be implemented in other ways, such as with programmable logic, electronic circuitry, or the like. In some alternative embodiments, certain of the operations may even be performed with limited human intervention. Moreover, these processes are not to be interpreted as exclusive of embodiments, but rather are provided as illustrative only.

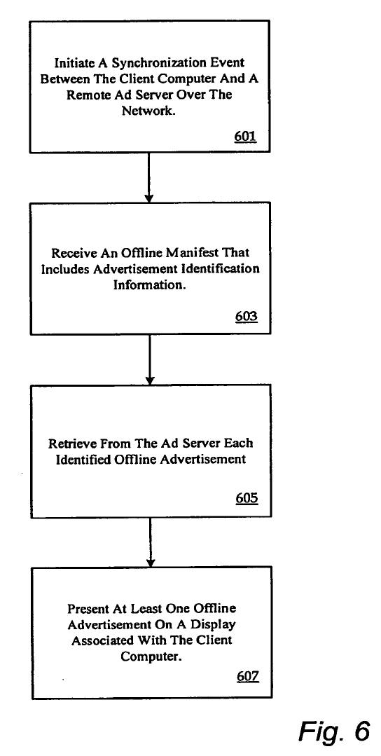

FIG. 6 is an operational flow diagram generally illustrating a process 600 for presenting an electronic advertisement on a client computer 121. The process begins at step 601, where a client computer 121 is online, meaning that the client computer 121 is coupled to a wide area network 101 to which other computing devices are also coupled, such as an advertisement server (ad server 111) and perhaps a content server.

At step 601, a synchronization event is initiated between the client computer 121 and a remote ad server 111 over the network 101. The ad server 111 includes advertisements that are intended for presentation in connection with content. For example, the content may include articles or other information for presentation on the client computer 121. The advertisements may include text or media that may be presented in conjunction with the content for the purpose of advertising, or the like. The synchronization event being for the purpose to making the client computer 121 ready for offline viewing.

At step 603, an offline manifest 214 that includes advertisement identification information is received. The advertisement identification information identifies one or more of the advertisements on the ad server 111 as offline advertisements. The identification can include a location identifier, dimensional characteristics, and any other information about the advertisements. In certain implementations, the offline manifest 214 may include the advertisements themselves.

At step 605, each offline advertisement identified in the offline manifest 214 is retrieved from the ad server 111. For instance, during a synchronization event, the client computer 121 may walk through the offline manifest 214 and issue a request to retrieve each advertisement identified in the manifest. The retrieved advertisements are stored locally for later offline use.

At step 607, while the client computer 121 is offline, at least one offline advertisement is presented on a display 310 associated with the client computer 121. In one example, a user of the client computer 121 makes use of content browsing software to view or access locally-stored content. In conjunction with displaying the content, the client computer 121 presents an advertisement selected from the advertisements stored locally at step 605. As mentioned above, selecting the appropriate advertisement may be based on various selection criteria, such as display 310 characteristics and the available placement opportunity, such as “white space” or the like.

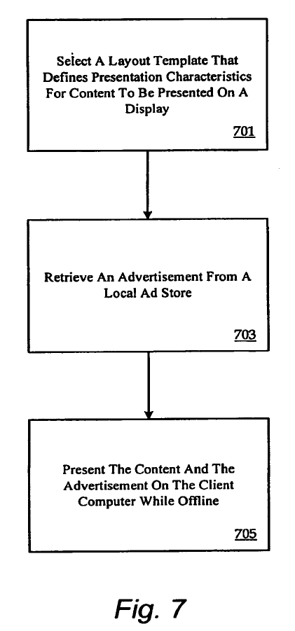

FIG. 7 is an operational flow diagram generally illustrating a process 700 for presenting an advertisement on a client computer 121. The process 700 may be performed on a client computer 121 that is configured for offline access of locally-cached content and locally-cached advertisements.

At step 701, a layout template is selected that defines presentation characteristics for content to be presented on a display 310. In this implementation, the selection is based on selection criteria about the content and the display 310. For example, the content being displayed may lend itself to a particular layout, such as a two column layout or a three column layout with a space for an image or an advertisement. In another implementation, the layout template selection may be driven or guided by a local ad server 416 that is configured to promote one or more advertisements, such as for business purposes, or the like.

At step 703, an advertisement is retrieved from a local ad store 312 in which reside a plurality of advertisements that have been made available for presentation on the client computer 121 while offline. Retrieving the advertisement may include selecting an appropriate advertisement based on the layout template selected at step 701. Additional information may also be used to identify the proper advertisement for presentation, such as empirical information about the content being presented, and the like.

At step 705, the content and the advertisement are presented on the client computer 121 while offline using the selected layout template. In accordance with the dynamic layout environment, the content and the advertisement are presented on the display 310 in a visually appealing manner.

Although the processes shown in FIGS. 6 and 7 are illustrated and described sequentially in a particular order, in other embodiments, the operations described in the blocks may be performed in different orders, multiple times, and/or in parallel. Further, in some embodiments, one or more operations described in the blocks may be separated into another block, omitted or combined.

Reference has been made throughout this specification to “one embodiment,” “an embodiment,” or “an example embodiment” meaning that a particular described feature, structure, or characteristic is included in at least one embodiment. Thus, usage of such phrases may refer to more than just one embodiment. Furthermore, the described features, structures, or characteristics may be combined in any suitable manner in one or more embodiments.

One skilled in the relevant art may recognize, however, that embodiments may be practiced without one or more of the specific details, or with other methods, resources, materials, etc. In other instances, well known structures, resources, or operations have not been shown or described in detail merely to avoid obscuring aspects of the embodiments.

While example embodiments and applications have been illustrated and described, it is to be understood that the invention is not limited to the precise configuration and resources described above. Various modifications, changes, and variations apparent to those skilled in the art may be made in the arrangement, operation, and details of the methods and systems disclosed herein without departing from the scope of the claimed invention.

What is claimed is:

1. A computer-implemented method for presenting an electronic advertisement on a client computer, the method comprising:
initiating a synchronization event between the client computer and a remote ad server, the ad server including advertisements for presentation in connection with content;
receiving an offline manifest that includes advertisement identification information, the advertisement identification information identifying one or more of the advertisements on the ad server as offline advertisements;
retrieving from the ad server each identified offline advertisements; and
presenting at least one offline advertisement on a display associated with the client computer while the client computer is offline.

2. The computer-implemented method recited in claim 1, wherein the offline manifest comprises a markup language document.

3. The computer-implemented method recited in claim 1, wherein retrieving the identified offline advertisements comprises retrieving the advertisements and storing the advertisements in a local ad store on the client computer.

4. The computer-implemented method recited in claim 1, wherein presenting the offline advertisement on the display further comprises selecting a layout template that defines presentation characteristics for content to be presented on a display.

5. The computer-implemented method recited in claim 4, wherein selecting the layout template is based on selection criteria about the content and the display.

6. The computer-implemented method recited in claim 5, wherein the selection criteria comprises current display characteristics of the display, characteristics of the content being presented, and whether an advertisement should be presented.

7. The computer-implemented method recited in claim 1, further comprising synchronizing the client computer to the ad server upon the client computer returning online.

8. The computer-implemented method recited in claim 7, wherein synchronizing the client computer comprises transferring usage data collected at the client computer while offline to the ad server.

9. The computer-implemented method recited in claim 8, wherein transferring the usage data comprises issuing a request for an advertisement to the ad server a number of times substantially equal to a number of times the advertisement was presented at the client computer while the client computer was offline.

10. A computer-readable medium encoded with computer-executable instructions for performing the computer-implemented method recited in claim 1.

11. A computer-implemented method for presenting an advertisement on a client computer, the method comprising:
selecting a layout template that defines presentation characteristics for content to be presented on a display, the selection being based on selection criteria about the content and the display;
retrieving an advertisement from a local ad store in which reside a plurality of advertisements that have been made available for presentation on the client computer while offline; and
presenting the content and the advertisement on the client computer while offline using the selected layout template.

12. The computer-implemented method recited in claim 11, wherein the selection criteria comprises current display characteristics of the display, characteristics of the content being presented, and whether an advertisement should be presented.

13. The computer-implemented method recited in claim 11, further comprising synchronizing the client computer to an ad server upon the client computer returning online.

14. The computer-implemented method recited in claim 13, wherein synchronizing the client computer comprises transferring to the ad server usage data collected at the client computer while offline.

15. The computer-implemented method recited in claim 14, wherein transferring the usage data comprises issuing a request for an advertisement to the ad server a number of times substantially equal to a number of times the advertisement was presented at the client computer while the client computer was offline.

16. The computer-implemented method recited in claim 11, wherein selecting the layout template is further based, at least in part, on a preference for a particular advertisement to be presented.

17. A computer-readable medium encoded with computer-executable instructions for performing the computer-implemented method recited in claim 11.

18. A computer-readable medium encoded with a data structure, comprising:
an offline manifest that includes identification information that identifies a plurality of advertisements that may be presented on a client computer while the client computer is offline, the offline manifest further comprising characteristic information about each advertisement in the plurality of advertisements, the characteristic information being useful in determining which layout template from among a plurality of layout templates to use to present the advertisement.

19. The computer-readable medium recited in claim 18, wherein the offline manifest further comprises at least one advertisement.

20. The computer-readable medium recited in claim 18, wherein the offline manifest comprises a markup language document.


SEQUENCE LISTING (18 sequences)

SEQ ID NO: 1 - DNA, 28 bp, Artificial Sequence
  Completely Synthetic DNA Sequence
atcagcaagaattaccattccagacatc

SEQ ID NO: 2 - DNA, 28 bp, Artificial Sequence
  Completely Synthetic DNA Sequence
catacaacttttcttccattgatagccc

SEQ ID NO: 3 - DNA, 19 bp, Artificial Sequence
  Completely Synthetic DNA Sequence
gttacttggcaccccagga

SEQ ID NO: 4 - DNA, 27 bp, Artificial Sequence
  Completely Synthetic DNA Sequence
catccaatttggtaaagaatcgtgtca

SEQ ID NO: 5 - DNA, 17 bp, Artificial Sequence
  Completely Synthetic DNA Sequence
amtcctcacagaacccc

SEQ ID NO: 6 - DNA, 26 bp, Artificial Sequence
  Completely Synthetic DNA Sequence
gcacattcaatccaatttggacttct

SEQ ID NO: 7 - DNA, 28 bp, Artificial Sequence
  Completely Synthetic DNA Sequence
catccaatttggtaaagaatcgtgtcat

SEQ ID NO: 8 - DNA, 17 bp, Artificial Sequence
  Completely Synthetic DNA Sequence
cagtgcttctagaaccc

SEQ ID NO: 9 - DNA, 24 bp, Artificial Sequence
  Completely Synthetic DNA Sequence
tgcatccaatttggtaaagaatcg

SEQ ID NO: 10 - DNA, 62 bp, Artificial Sequence
  Completely Synthetic DNA Sequence
ttactggcttatcgaaattaatacgactcactatagggaggagtcatggcagtgccctttgt

SEQ ID NO: 11 - DNA, 64 bp, Artificial Sequence
  Completely Synthetic DNA Sequence
tagaaggcacagtcgaggctgatcagcgggtttaaactcatgcatccaatttggtaaagaatcg

SEQ ID NO: 12 - DNA, 80 bp, Artificial Sequence
  Completely Synthetic DNA Sequence
aatccaatttggacttctctccagtaaacagtgcttctagaacccctggcagcggttggtcaaaagaatgacacgattct

SEQ ID NO: 13 - DNA, 80 bp, Artificial Sequence
  Completely Synthetic DNA Sequence
agaatcgtgtcattcttttgaccaaccgctgccaggggttctagaagcactgtttactggagagaagtccaaattggatt

SEQ ID NO: 14 - DNA, 954 bp, Artificial Sequence
  Completely Synthetic DNA Sequence
atggcagtgccctttgtggaagactgggacttggtgcaaaccctgggagaaggtgcctatggagaagttcaacttgctgtgaatagagtaactgaagaagcagtcgcagtgaagattgtagatatgaagcgtgccgtagactgtccagaaaatattaagaaagagatctgtatcaataaaatgctaaatcatgaaaatgtagtaaaattctatggtcacaggagagaaggcaatatccaatatttatttctggagtactgtagtggaggagagctttttgacagaatagagccagacataggcatgcctgaaccagatgctcagagattcttccatcaactcatggcaggggtggtttatctgcatggtattggaataactcacagggatattaaaccagaaaatcttctgttggatgaaagggataacctcaaaatctcagactttggcttggcaacagtatttcggtataataatcgtgagcgtttgttgaacaagatgtgtggtactttaccatatgttgctccagaacttctgaagagaagagaatttcatgcagaaccagttgatgtttggtcctgtggaatagtacttactgcaatgctcgctggagaattgccatgggaccaacccagtgacagctgtcaggagtattctgactggaaagaaaaaaaaacatacctcaacccttggaaaaaaatcgattctgctcctctagctctgctgcataaaatcttagttgagaatccatcagcaagaattaccattccagacatcaaaaaagatagatggtacaacaaacccctcaagaaaggggcaaaaaggccccgagtcacttcaggtggtgtgtcagagtctcccagtggattttctaagcacattcaatccaatttggacttctctccagtaaacagtgcttctagaacccctggcagcggttggtcaaaagaatga

SEQ ID NO: 15 - protein, 317 aa, Artificial Sequence
  Completely Synthetic Amino Acid Sequence
MAVPFVEDWDLVQTLGEGAYGEVQLAVNRVTEEAVAVKIVDMKRAVDCPENIKKEICINKMLNHENVVKFYGHRREGNIQYLFLEYCSGGELFDRIEPDIGMPEPDAQRFFHQLMAGVVYLHGIGITHRDIKPENLLLDERDNLKISDFGLATVFRYNNRERLLNKMCGTLPYVAPELLKRREFHAEPVDVWSCGIVLTAMLAGELPWDQPSDSCQEYSDWKEKKTYLNPWKKIDSAPLALLHKILVENPSARITIPDIKKDRWYNKPLKKGAKRPRVTSGGVSESPSGFSKHIQSNLDFSPVNSASRTPGSGWSKE

SEQ ID NO: 16 - DNA, 40 bp, Artificial Sequence
  Completely Synthetic DNA Sequence
cccggaattcaccatggcagtgccctttgtggaagactgg

SEQ ID NO: 17 - DNA, 54 bp, Artificial Sequence
  Completely Synthetic DNA Sequence
tgtgtccggccgtcagtgatggtgatggtgatgttcttttgaccaaccgctgcc

SEQ ID NO: 18 - protein, 13 aa, Artificial Sequence
  Completely Synthetic Amino Acid Sequence
GGRARTSSFAEPG